(19) United States
(12) Patent Application Publication
(10) Pub. No.: US 2012/0142050 A1
(43) Pub. Date: Jun. 7, 2012

(54) NOVEL EXPRESSION VECTOR

(75) Inventors: Eitaro Matsumura, Ishikawa (JP); Momoko Hamasaki, Hyogo (JP); Akinobu Matsuyama, Niigata (JP)

(73) Assignee: Daicel Chemical Industries, Ltd, Osaka (JP)

(21) Appl. No.: 13/144,176

(22) PCT Filed: Jan. 13, 2010
(86) PCT No.: PCT/JP2010/050245

§ 371 Date: Sep. 20, 2011

(30) Foreign Application Priority Data
Jan. 13, 2009 (JP) 2009-004905

(51) Int. Cl. C12N 1/21; C12P 21/00; C12P 1/04; C12N 15/74

(52) U.S. Cl. 435/69.1; 435/320.1; 435/252.3; 435/170

(57) ABSTRACT

The present inventors constructed a transformation system which uses as a host the Kocuria rhizophila NBRC 103217 strain which can maintain its cellular structure in various organic solvents and shows little protein leakage.

As a result, by developing novel shuttle vectors derived from Kocuria varians NBRC 15358, the present inventors successfully induced the expression of a foreign protein in microorganisms belonging to the genus Kocuria which can maintain their cellular structure in non-aqueous systems and show little protein leakage.

### TECHNICAL FIELD

The present invention relates to shuttle vectors that are replicable and allow inducible expression of a foreign gene in microorganisms belonging to the genus Kocuria, and uses thereof.

### BACKGROUND ART

Processes for producing useful substances such as industrial materials using microorganisms generally have the characteristic of low energy consumption and less waste generation. Therefore, with concerns about exhaustion of resources and effects of emissions such as CO₂ on the environment in recent years, development of techniques for producing useful substances by utilizing the functions of microorganisms (bioprocesses) is needed as a basic manufacturing technology platform in environment-conscious recycling-oriented industrial systems.

To construct fossil resource-independent or environmentally-friendly production processes in the chemical industry with the ultimate aim of carbon neutrality, it is necessary to construct a chemical production system that includes a bio-refinery from biomass. To accomplish this, several technical breakthroughs are necessary, and in particular, it is necessary to expand the range of use and application range of bioprocesses that use microbial cells which are suitable for natural resource recycling. Currently, bioprocesses are basically performed in an aqueous reaction site, and this significantly narrows down the application range of bioprocesses because producible targets (chemicals) become limited. While the aqueous reaction site is suitable for conversion and production of hydrophilic substances, it is not suitable for conversion, reaction, or production of hydrophobic substances which account for a large segment of chemicals.

Consequently, techniques are essential for designing a microbial factory or a biocatalyst by freely coordinating an optimal enzyme gene and host cell, which may produce chemicals of interest in an optimal reaction site. There are few examples of studies relating to the functions of microbial reactions in a non-aqueous reaction site containing organic solvents and examples of industrial production in such reaction site. A microorganism that can be practically used for microbial conversion reactions in organic solvents is the microorganism Kocuria rhizophila NBRC 103217, as searched from the standpoint of maintaining cellular structure in organic solvents, and its genetic information has been elucidated (Non-patent Document 1). However, many sections including the microorganism's mechanism for durability are unknown.

To construct a microbial factory or a biocatalyst that has more durability in a non-aqueous reaction site, it is necessary to elucidate the mechanism for durability and design host cells based on that mechanism in parallel.

On the other hand, with the focus on enzyme reactions, the most important factor to achieve a cost-competitive production process is a reaction that produces an expensive product of high value from inexpensive raw materials, and enzymes that catalyze such reactions are required. Furthermore, in the production step of various chemicals, practical enzymes that catalyze reactions useful in producing functional groups necessary for conversion into chemicals with higher value are also required. Reactions that produce expensive products from inexpensive raw materials include carbon-to-carbon binding reactions. For example, it is thought that reactions that polymerize through carbon-to-carbon binding by an aldolase using inexpensive low-molecular weight compounds such as an aldehyde as the starting material and reactions that polymerize through fixation of inexpensive carbon dioxide gas (carbon dioxide fixation reaction) are useful.

Furthermore, when various chemicals are produced, reactions useful for production of functional groups necessary to convert into those chemicals include, for example, hydroxylation reaction and carbon oxygenation reaction. In the case of these reactions, since chemical substances that do not dissolve in water such as hydrocarbons are substantively used as the starting material in most cases, it is necessary to carry out the reaction in a non-aqueous reaction site, and enzymes that can be used in a non-aqueous system become necessary. However, the relationship between organic solvent resistance and enzyme including three-dimensional structure is not clear, and the relationship needs to be elucidated by using organic solvent-resistant enzymes obtained so far (for example, nitrilase) as models and observing their three-dimensional structure and changes in organic solvent resistance by mutations.

Accordingly, for smooth progression of microbial reactions in a non-aqueous reaction site, development of vectors capable of expressing a desired foreign protein (organic solvent-resistant enzyme) in a microorganism that maintains the cellular structure even in a non-aqueous system has been desired.

### PRIOR ART DOCUMENTS

### Non-patent Document

[Non-patent Document 1] J. Bacteriol. Vol. 190, p 4139-4146, 2008.

### DISCLOSURE OF THE INVENTION

### Problems to be Solved by the Invention

The present invention was achieved in view of the above circumstances. An objective of the present invention is to provide shuttle vectors that are replicable and allow inducible expression of a foreign protein in microorganisms belonging to the genus Kocuria.

### Means for Solving the Problems

To solve the above-mentioned problems, the present inventors constructed a transformation system which uses as a host the Kocuria rhizophila NBRC 103217 strain which maintains its cellular structure in various organic solvents and show little protein leakage.

First, the entire nucleotide sequence (2.7 Mb) of the Kocuria rhizophila NBRC 103217 genome was determined, and 2,356 ORFs were estimated from the nucleotide sequence. Based on the ORF information, the upstream and downstream sequences of the SD sequence that are most suitable for translation were predicted.

Next, a transformation system of the Kocuria rhizophila NBRC 103217 strain was established using the EZ-TN5<KAN-2>Tnp transposon. It was found out that the efficiency of transformation is greatly improved by using the NBRC 10317 strain bacterial cells grown until OD600=0.7 in an LB medium containing 1.5% glycine as the competent cells, and after incubation at 35° C. for five minutes, they are used for electroporation. Furthermore, when a transposon containing not only a kanamycin resistance gene which is a selection marker but also the GFP gene was constructed and the Kocuria rhizophila NBRC 103217 strain was transformed, a transformant carrying the GFP gene could be obtained even though the transformation efficiency was 3% of that when a transposon carries only the kanamycin resistance gene.

Next, since a rescue plasmid of the Kocuria varians NBRC 15358-derived plasmid (pKV15358-1) is replicable in the Kocuria rhizophila NBRC 103217 strain, to produce a plasmid vector that is replicable in the Kocuria rhizophila NBRC 103217 strain, the pKV15358-3 vector (4.3 kb) for the Kocuria rhizophila NBRC 103217 strain was constructed by miniaturizing pKV15358-1 and adding the above-mentioned optimal-for-translation upstream and downstream sequences of the SD sequence in the Kocuria rhizophila NBRC 103217 strain. When a nitrilase gene derived from the Arthrobacter sp. F73 strain was expressed in the Kocuria rhizophila NBRC 103217 strain, nitrilase activity was shown even though the expression level was about 1% of that in Escherichia coli. When the same transformant was used in an ethyl acetate-water biphasic reaction in which mandelic acid production is not observed in E. coli, even in a system containing 60% ethyl acetate, it showed a 60% activity of that in an aqueous-phase reaction.

Furthermore, to obtain a promoter for increasing the expression level of a foreign gene, superoxide dismutase was discovered as one of the proteins highly produced in the stationary phase of culture. It was revealed that expression of the nitrilase gene was increased 150 times when a promoter region that expresses the superoxide dismutase-encoding gene was identified, cloned, and introduced into a vector for the Kocuria rhizophila NBRC 103217 strain.

More specifically, by developing novel shuttle vectors, the present inventors successfully induced expression of a foreign protein in microorganisms of the genus Kocuria which can maintain their cellular structure in non-aqueous systems and show little protein leakage, and thereby completed the present invention.

More specifically, the present invention provides:

[1] a vector capable of expressing a foreign protein in a microorganism, which comprises a polynucleotide comprising the nucleotide sequence of SEQ ID NO: 1, or a polynucleotide that hybridizes under stringent conditions with a complementary strand of a polynucleotide comprising the nucleotide sequence of SEQ ID NO: 1;
[2] the vector of [1], wherein the vector additionally comprises a polynucleotide comprising the nucleotide sequence of SEQ ID NO: 2, or a polynucleotide that hybridizes under stringent conditions with a complementary strand of a polynucleotide comprising the nucleotide sequence of SEQ ID NO: 2;
[3] the vector of [1] or [2], wherein the vector additionally comprises a polynucleotide comprising the nucleotide sequence of SEQ ID NO: 3 or 4, or a polynucleotide that hybridizes under stringent conditions with a complementary strand of a polynucleotide comprising the nucleotide sequence of SEQ ID NO: 3 or 4;
[4] the vector of any one of [1] to [3], wherein the microorganism in which a foreign protein is expressed is a microorganism belonging to the genus Kocuria, the genus Micrococcus, or the genus Arthrobacter;
[5] the vector of [4], wherein the microorganism belonging to the genus Kocuria is the Kocuria rhizophila NBRC 103217 strain;
[6] a recombinant vector in which a DNA encoding a foreign protein is inserted into the vector of any one of [1] to [5];
[7] a transformant in which a host is transformed with the vector of any one of [1] to [5] and the recombinant vector of [6];
[8] a method for producing a foreign protein, which comprises the steps of culturing the transformant of [7] and isolating a foreign protein from the obtained culture; and
[9] a method for producing a substance of interest, wherein the transformant of [7] is used as a biocatalyst for producing the substance of interest in a non-aqueous reaction site.

### BRIEF DESCRIPTION OF THE DRAWINGS

FIG. 1 shows the category scores.

FIG. 2 shows the results of analyzing the distribution of all ORFs of the NBRC 103217 strain.

FIG. 3 depicts a two-dimensional electrophoresis.

FIG. 4 shows the results of MALDI-TOF MS.

### MODE FOR CARRYING OUT THE INVENTION

The present invention provides shuttle vectors that are replicable and allow inducible expression of a foreign protein in microorganisms belonging to the genus Kocuria.

Vectors of the present invention carry a DNA region that is replicable in microorganisms of the genus Kocuria, and a drug marker that confers drug resistance to microorganisms of the genus Kocuria. Vectors of the present invention are shuttle vector plasmids that can be replicated in microorganisms of the genus Kocuria, and are efficient vectors for expressing a foreign protein in microorganisms of the genus Kocuria.

Shuttle vectors of the present invention include circular plasmids that can be obtained from the Kocuria varians NBRC15358 strain. By elucidating the DNA region involved in replication of these plasmids and producing plasmids containing that DNA region, such plasmids are replicable in microorganisms of the genus Kocuria.

The replicable DNA region in microorganisms belonging to the genus Kocuria is not particularly limited as long as it is a region that enables a vector to be replicated when a vector containing the region is introduced into a microorganism of the genus Kocuria, and includes, for example, an autonomously replicating region derived from an endogenous plasmid vector pKV15358-1 (SEQ ID NO: 5) carried by the Kocuria varians strain. The autonomously replicating region derived from pKV15358-1 may be the full-length pKV15358-1, but to simplify the genetic manipulations it is preferably a portion of pKV15358-1. A portion of pKV15358-1 includes a region comprising the nucleotide sequence of SEQ ID NO: 1. Furthermore, as long as it can function in microorganisms of the genus Kocuria, it may be a DNA region comprising a sequence with one or several nucleotide substitutions, deletions, insertions, and/or such in the nucleotide sequence of SEQ ID NO: 1, or a DNA region that hybridizes under stringent conditions with a polynucleotide comprising the nucleotide sequence of SEQ ID NO: 1 or a probe that may be prepared from this sequence. Herein, “several” refers to, for example, two to 50, preferably two to ten, and more preferably two to five. Stringent conditions include washing conditions for conventional Southern hybridization, which are 60° C. and a salt concentration corresponding to 1×SSC, and 0.1% SDS, or preferably 0.1×SSC, and 0.1% SDS. One or several nucleotide substitutions, deletions, and/or insertions can be carried out by techniques known to those skilled in the art at the time of filing the application.

The above-described region necessary for autonomous replication in the pKV15358-1 vector can be identified by deletion experiments which use expression of an antibiotic resistance gene in antibiotic-sensitive host cells as an indicator. That is, after introducing a drug resistance gene such as a kanamycin resistance gene or an ampicillin resistance gene into the pKV15358-1 vector, the vector is digested with a single restriction enzyme or a combination of restriction enzymes that do not digest this gene, recircularized by a DNA ligase, and then used to transform drug-sensitive bacterial cells. Drug-resistant bacterial strains that are selected from these cells using drug resistance as the indicator carry the derived vector containing the autonomously replicating region of the pKV15358-1 vector; and the autonomously replicating region of the pKV15358-1 vector can be identified by comparing the restriction enzyme map of the vector.

In the present invention, a drug marker that confers resistance to microorganisms of the genus Kocuria is not particularly limited as long as it is a gene that confers drug resistance when it is introduced into the bacteria, and includes, for example, a kanamycin resistance gene, an ampicillin resistance gene, a neomycin resistance gene, or a chloramphenicol resistance gene.

Plasmid vectors of the present invention are vectors comprising the above-mentioned autonomously replicating region and a drug resistance marker, and such vectors specifically include pKV15358-2 (SEQ ID NO: 6). pKV15358-2 is a vector composed of a fragment comprising an autonomously replicating region obtained by cleaving the above-mentioned pKV15358-1 with the XbaI restriction enzyme, as well as a kanamycin resistance gene and such. The vector of the present invention may be a modified form of pKV15358-2. Examples of the modification include mutations at sites not involved in the replication ability or drug resistance, and deletions or substitutions of a portion of the restriction enzyme sites.

A vector of the present invention may further comprise an SD sequence derived from a microorganism belonging to the genus Kocuria. The SD sequence is not particularly limited, but is preferably, for example, the SD sequence of SEQ ID NO: 2. Vectors further comprising an SD sequence include pKV15358-3 (SEQ ID NO: 7).

Furthermore, a promoter that may function in microorganisms of the genus Kocuria can be further incorporated. The promoter is not particularly limited, and examples include a promoter that expresses a gene encoding superoxide dismutase.

In the present invention, microorganisms belonging to the genus Kocuria are not particularly limited, but a preferred example includes Kocuria rhizophila NBRC 103217.

The present invention relates to transformants produced by transforming a host with a shuttle vector for inducible expression in microorganisms of the genus Kocuria into which a foreign protein-encoding DNA is inserted.

Furthermore, the present invention relates to methods for producing a foreign protein, which comprise the steps of culturing the above-mentioned transformants and isolating a foreign protein from the obtained culture.

To express a foreign protein that one desires to express in microorganisms of the genus Kocuria using the present invention's shuttle vector for inducible expression in microorganisms of the genus Kocuria, it is necessary to produce a recombinant vector into which a DNA encoding the foreign protein is inserted into a vector of the present invention.

Recombinant vectors of the present invention are produced by inserting a DNA encoding a foreign protein into the vectors of the present invention (for example, pKV15358-2 or pKV15358-3).

In the present invention, the foreign protein that one desires to express is not particularly limited, and includes enzymes, hormones, cytokines, and prepared proteins. Preferred examples of the foreign protein of the present invention include a catalytic enzyme used when producing substances in non-aqueous systems (for example, in organic solvents). An example of the catalytic enzyme includes nitrilase and the vector into which the foreign protein is introduced includes pKV15358-4 (SEQ ID NO: 20).

A transformant of the present invention can be produced by transforming a host with a vector of the present invention or a recombinant vector of the present invention. For example, the electroporation method, calcium phosphate method, and DEAE dextran method can be used as the method for transformation. As the host for expressing a foreign protein or host to be used for propagation and collection of vector plasmids, microorganisms of the genus Kocuria can be used.

The present invention enables production of a foreign protein by culturing the above-described transformant and isolating the foreign protein from the obtained culture. As the method for culturing the transformant, one can appropriately select a method suitable for the microorganism of the genus Kocuria used as the host. In the method, culturing can be carried out in a non-aqueous system (for example, in an organic solvent).

Expression of a foreign protein can be induced by the vector of the present invention using inducers (for example, ε-caprolactam and isovaleronitrile).

In the present invention, “culture” refers to a material that can be obtained by culturing such as bacterial cells, culture, cell-free extract, and cell membrane. Cell-free extracts can be obtained, for example, by physically disrupting cultured bacterial cells using a homogenizer after adding a sodium phosphate buffer, then separating by centrifugation and collecting the supernatant so that undisrupted bacterial cells (cells) are not present. Cell membrane can be obtained by suspending the pellet obtained by the above-mentioned centrifugation in a solubilizing buffer.

For a foreign protein of interest, a culture solution may be used as it is. Alternatively, a foreign protein of interest may be concentrated and purified using known methods such as dialysis and ammonium sulfate precipitation, or known methods such as various chromatographic methods such as gel filtration, ion exchange, and affinity chromatography alone or in combination.

Furthermore, the present invention relates to a method for producing a substance of interest, wherein the above-mentioned transformant is used as a biological catalyst for producing the substance of interest in a non-aqueous reaction site.

Non-aqueous system refers to a reaction site that is not 100% water, a reaction site having an arbitrary ratio of organic solvent and water, a reaction site which is a supercritical gas, or a reaction site which is an ionic liquid. Microorganisms of the genus Kocuria are microorganisms that maintain their cellular structure even in a non-aqueous system. Even when a chemical substance serving as a starting material of the substance of interest is contacted with a transformant of a microorganism of the genus Kocuria (used as a biocatalyst) in a non-aqueous reaction site, activity is not lost due to dissolution of the transformant and the substance of interest can be obtained with high productivity. In the present invention, the substance of interest is not particularly limited, and examples include mandelic acid and such.

All prior art documents cited in this specification are incorporated herein by reference.

### EXAMPLES

### Example 1

### Estimation of Upstream and Downstream Sequences of the SD Sequence Suitable for the Kocuria rhizophila NBRC 103217 Strain

The entire nucleotide sequence (2.7 Mb) of the Kocuria rhizophila NBRC 103217 strain genome was determined, and 2,356 ORFs were estimated from that nucleotide sequence. Based on the ORF information, the upstream and downstream sequences of the SD sequence that are most suitable for translation were predicted.

Five nucleotides upstream and nine nucleotides downstream of each of the start codons for all ORFs (2,356 ORFs) predicted from the whole genome sequence (2,697,540 bp) of the Kocuria rhizophila NBRC 103217 strain were analyzed by quantification theory type 2. In the analysis, as pseudo start codon data, codons ATC; GTG, and TTG which do not overlap with start codons were extracted with a frequency equivalent to that of the start codons.

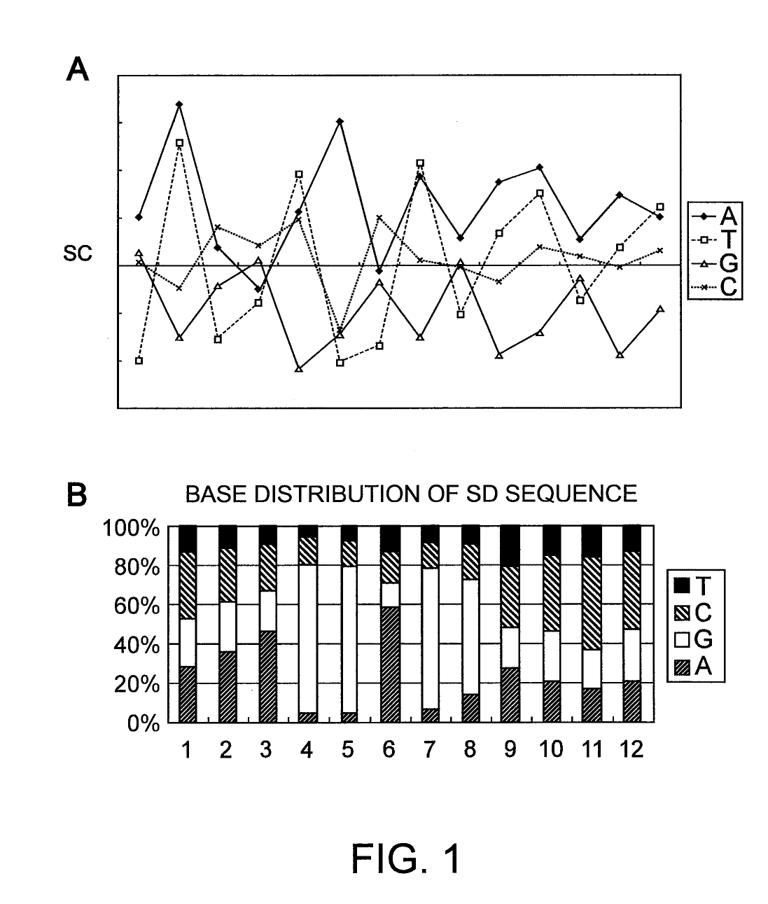

Since the category scores (FIG. 1) indicate importance of each nucleotide, “AACCT” (SEQ ID NO: 8), which is a sequence of nucleotides showing high scores at each position, was predicted to be the most suitable sequence immediately upstream of the start codon for translation.

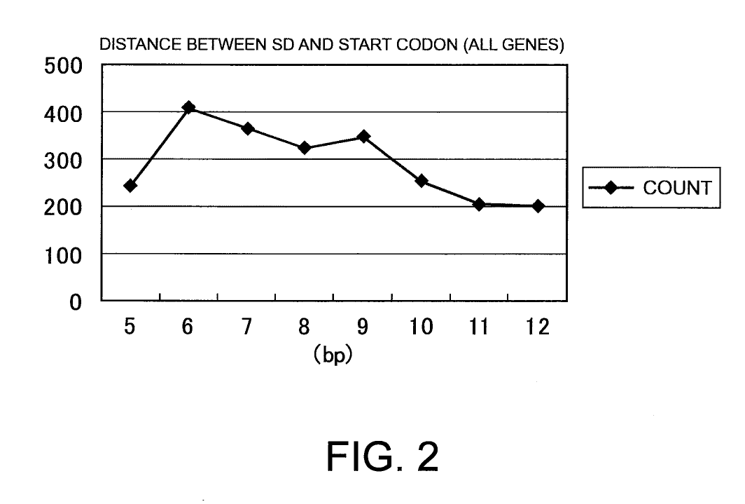

Furthermore, since the sequence of the 3′ end of 16S rRNA is AUCACCUCCUUUCU-3′ (SEQ ID NO: 9), the SD sequence was predicted to be AGGAGG (SEQ ID NO: 10). As shown in FIG. 2, as a result of analyzing the distribution of the predicted sequences upstream and downstream sequences of the SD sequence in all ORFs of the NBRC 103217 strain, this also confirmed that there is a strong complementarity to the 3′ end sequence of the 16S ribosomal RNA (nucleotide sequence of positions 3 to 7). The most frequent number of nucleotides in the sequence between the SD sequence and the start codon was 6 bp, but since 7 bp showed nearly the same value and 7 bp is often optimal in other microorganisms, 7 bp may be the optimal number of nucleotides between the SD sequence and the start codon.

Therefore, 5′-CTAGCAAGGAGGCCAACCT-3′ (SEQ ID NO: 11) was predicted as the optimal upstream and downstream sequences of the SD sequence by combining the “AACCT” sequence (SEQ ID NO: 8) upstream of the start codon estimated from quantification analysis type 2 with the results of distribution analysis of the upstream and downstream sequences of the SD sequence.

When introducing into an actual vector, an NdeI site which contains an ATG start codon was added to improve convenience at the time of gene introduction, and this was used as the final upstream and downstream sequences of the SD sequence (5′-CTAGCAAGGAGGCCAACATATG-3′, SEQ ID NO: 12).

Next, a transformation system of the Kocuria rhizophila NBRC 103217 strain was established using the EZ-TN5<KAN-2>Tnp transposon. It was found out that the efficiency of transformation is greatly improved by using the NBRC 10317 strain bacterial cells grown until OD600=0.7 in an LB medium containing 1.5% glycine as the competent cells, and after incubation at 35° C. for five minutes, they are used for electroporation. Furthermore, when a transposon containing not only a kanamycin resistance gene which is a selection marker but also the GFP gene was constructed and the NBRC 103217 strain was transformed, a transformant carrying the GFP gene could be obtained even though the transformation efficiency was 3% of that when a transposon carries only the kanamycin resistance.

### Example 2

### Introduction of a Plasmid Extracted from the Kocuria varians NBRC 15358 Strain into the NBRC 103217 Strain

Next, a plasmid vector that is replicable in the Kocuria rhizophila NBRC 103217 strain was prepared. Since the rescue plasmid (SEQ ID NO: 5) of the plasmid (pKV15358-1) derived from Kocuria varians NBRC 15358 is replicable in the Kocuria rhizophila NBRC 103217 strain, this plasmid was miniaturized and the upstream and downstream sequences of the SD sequence (SEQ ID NO: 2) found in Example 1 was further added to construct vector pKV15358-3 (4.3 kb) (SEQ ID NO: 7) for the Kocuria rhizophila NBRC 103217 strain.

More specifically, the Kocuria varians NBRC15358 strain was cultured while shaking at 150 rpm in a 500-mL baffled conical flask containing 100 mL of LB medium at 30° C. until the turbidity at 600 nm reached 8.0. Two-hundred milliliters of the culture solution was centrifuged (9,970×g, 4° C., ten minutes) to collect the cells, and after washing with 15 mL of STE (10 mM Tris-HCl, 0.5 mM EDTA, 5 mM NaCl; pH 8.0), the cells were collected by centrifugation (9,970×g, 4° C., ten minutes). From the obtained bacterial cells, plasmid DNA was extracted using the Qiagen Plasmid Maxi Kit (Qiagen). When using this kit, 4 mg/mL of lysozyme was added to Buffer P1, and after suspending the bacterial cells in this solution, this was incubated at 37° C. for two hours. Operations thereafter were carried out according to the method recommended by the manufacturer.

25 mM MgCl₂ was added to the obtained DNA solution (final 2 mM MgCl₂), and then RingMaster Nuclease (Novagen) was added to achieve 0.5 U/μL. The contaminating chromosomal DNAs were removed by one hour reaction at 37° C. After completion of the reaction, the enzyme was inactivated by incubation at 75° C. for ten minutes. Plasmid DNA was collected by ethanol precipitation, and dissolved in 20 μL of TE buffer (10 mM Tris-HCl, 1 mM EDTA; pH 8.0).

To the obtained plasmid, a transposon was inserted according to the instructions for the EZ-Tn5<R6Kγori/KAN-2> Insertion Kit (Epicentre). TransforMax EC100D pir Electrocompetent E. coli (Epicentre) was transformed using the reaction solution, and the transformants were selected on an LB agar plate containing 50 μg/mL kanamycin.

From the six strains of obtained transformants, plasmid DNAs were prepared and the NBRC 103217 strain was transformed. Competent cells of the NBRC 103217 strain were prepared using bacterial cells obtained by growing this bacterial strain in shake culture at 30° C. in a 500-mL baffled conical flask containing 100 mL of LB medium supplemented with 1.5% (w/v) glycine until the turbidity at 600 nm reached 0.7. The culture solution was centrifuged (1,400×g, 4° C., ten minutes) to collect the bacterial cells, and the cells were washed twice with 200 mL and once with 35 mL of cooled sterilized water. The cells were further washed twice with 35 mL of 10% (v/v) glycerol solution, and suspended in 10% (v/v) glycerol solution so that the turbidity at 600 nm becomes approximately 50. The obtained bacterial cell suspension was dispensed into a 1.5 mL tube (100 μL/tube), and after freezing using liquid nitrogen, this was stored at −80° C.

The competent cells were thawed on ice, 0.5 μg of DNA was added, and the NBRC 103217 strain was transformed using the electroporation method (800Ω, 25 μF, 12.5 kV/cm) using Gene Pulser (Bio-rad). After electroporation and addition of 0.9 mL SOC medium, it was cultured while shaking (30° C., 150 rpm, 16 hours). Then, colonies obtained by 30° C. culturing on an SOB agar plate containing 400 μg/mL kanamycin were selected.

Of the six types of plasmids tested for introduction into the NBRC 103217 strain, the plasmid of interest was retained in the cases where four types of plasmids were used.

### Example 3

### Introduction of the Arthrobacter sp. F73 Strain-Derived Nitrilase Gene into pKV15358-3

Using the pKV15358-3 vector constructed in Example 2, a nitrilase gene derived from the Arthrobacter sp. F73 strain was expressed in the Kocuria rhizophila NBRC 103217 strain.

To enable amplification of a DNA fragment with NdeI site added to the 5′ end and XbaI site added to the 3′ end of the nitrilase gene derived from Arthrobacter sp. F73 strain, two types of primers, F73Nit-309F-NdeI primer (5′-GTGCATATGAGCGAGTACACCCAGCAATACCGCGTC-3′, SEQ ID NO: 13) and F73Nit-1430R-XbaI primer (5′-TCCTCTAGATCAGAGCTGAACGGCCTCCTCCGAACGC-3′, SEQ ID NO: 14) were designed. PCR was performed by adding 37.5 U/mL of Pfu turbo DNA polymerase to a reaction system containing 0.2 μM of each primer, 1 ng/μL of chromosomal DNA of the F-73 strain as a template, and 2.5% (v/v) DMSO. The PCR included incubation at 98° C. for two minutes and thirty seconds, and then 30 cycles of a reaction consisting of three steps of 98° C. for 30 seconds, 55° C. for one minute, and 72° C. for one minute and thirty seconds, followed by incubation at 72° C. for ten minutes. The amplified DNA fragment was purified using the GFX PCR Purification Kit (GE Healthcare), and then digested with NdeI and XbaI, and ligated with plasmid pKV15358-3 digested with the same restriction enzymes. TransforMax EC100D pir Electrocompetent E. coli (Epicentre) was transformed with the obtained reaction solution, and transformants carrying the target pKV15358-4 plasmid were obtained from the strains grown in an LB medium containing 50 μg/mL kanamycin.

The NBRC 103217 strain was transformed using plasmid DNA extracted from these transformants to obtain NBRC 103217 strain transformants carrying these plasmids. For bacterial cells obtained by growing the strain in shake culture (250 rpm) at 20° C., 25° C., or 30° C. in an LB medium containing 300 μg/mL kanamycin, nitrilase activity was investigated by using mandelonitrile present in the cells as a substrate.

As a result, activity was not seen when grown at 30° C., but activity was observed when grown at 20° C. and 25° C. The highest activity was observed when cultured at 25° C. for 64 hours. For each bacterial cell, this was approximately 1% of that in E. coli (the activity of the NBRC 103217 strain bacterial cell suspension per OD₆₀₀=1 was 6.1 mU/mL).

### Example 4

### Production of Mandelic Acid in a Biphasic Reaction Site using the Kocuria Varians NBRC 103217 Strain

When the same transformant was used in an ethyl acetate-water biphasic reaction in which mandelic acid production is not observed in E. coli, even in a system containing 60% ethyl acetate, it showed a 60% activity of that in an aqueous phase reaction. More specifically, the following examinations were carried out.

The NBRC 103217 strain carrying pKV15358-5 (SEQ ID NO: 21) was cultured while shaking (150 rpm) for 144 hours in 100 mL of LB medium containing 300 μg/mL kanamycin (500-mL baffled conical flask), and the obtained culture solution (OD₆₀₀=12.6) was centrifuged (2,300×g, 4° C., ten minutes) to collect bacterial cells. Bacterial cells were suspended in 50 mM potassium phosphate buffer (pH 8.0), and bacterial cells were reacted with mandelonitrile as the substrate.

The total volume (0.5 mL) of the reaction solution was prepared at 50% (v/v) ethyl acetate, 1 M mandelonitrile, and 50 mM potassium phosphate buffer (pH 8.0), and the bacterial cells were added so that OD₆₀₀=10. The reaction was initiated by adding the bacterial cell suspension. Using a BioShaker M-BR-022UP (Taitec), the reaction solution was incubated at 30° C. for two hours while shaking at 1,400 rpm. Then, the reaction system was homogenized by adding 0.5 mL of methanol, and centrifuged (20,400×g, 4° C., ten minutes) to remove bacterial cells. The amount of mandelic acid contained in the obtained supernatant was quantified by HPLC. For the apparatus, LC-10ADvp (SHIMADZU) was used, and for the column, the Inertsil ODS-3 column (4) 4.6×150 mm, GL Sciences) was used. Analyses were carried out by setting the column temperature to 40° C., and passing 10 mM phosphoric acid-acetonitrile (55:45) as the eluent at a flow rate of 1 mL/min. The standard curve was produced using 0.02, 1, 5, 10, 15, 20, and 50 mM R-mandelic acid. The retention time was 2.2 minutes. The samples after completion of the bacterial cell reaction were diluted ten-fold using the eluent and subjected to HPLC. In the reaction using the K rhizophila NBRC 103217 (pKV15358-5) bacterial cells in a solvent system of water/ethyl acetate=1/1, 83.7 g/L of mandelic acid accumulated.

### Example 5

### Searching a Promoter Highly Expressed in the Kocuria rhizophila NBRC 103217 Strain

Furthermore, to obtain a promoter for increasing the expression level of a foreign gene, superoxide dismutase was discovered as one of the proteins highly produced in the stationary phase of culture. It was revealed that expression of the nitrilase gene was increased 150 times when a promoter region that expresses the superoxide dismutase-encoding gene was identified, cloned, and introduced into a vector for the Kocuria rhizophila NBRC 103217 strain. More specifically, the following examinations were carried out.

The Kocuria rhizophila NBRC 103217 strain was cultured while shaking at 150 rpm at 30° C. in a 500-mL baffled conical flask containing 100 mL of LB medium until the turbidity at 600 nm reached 11 (65 hours). 20 mL of the culture solution was centrifuged (2,300×g, 4° C., ten minutes) to collect bacterial cells, the cells were washed with 5 mL of STE (10 mM Tris-HCl, 0.5 mM EDTA, 5 mM NaCl; pH 8.0), and then centrifuged (2,300×g, 4° C., ten minutes) to collect the cells. The obtained bacterial cells were stored at −30° C.

Bacterial cells in 10 mL of culture solution were suspended in 0.7 mL of solubilizing buffer (10 mM Tris-HCl, 8 M urea, 4% (w/v) CHAPS, 60 mM DTT; pH 7.4), transferred to a 2-mL tube containing 0.5 mL of glass beads (1) 0.1 mm), and then the cells were disrupted using a Multi-Beads shocker (Yasui Kikai) (2,500 rpm, 4° C., 20 minutes). Supernatant obtained from centrifugation (20,400×g, 4° C., ten minutes) was collected. Protein was purified from 100 μl, of the obtained supernatant using the ReadyPrep 2-D Cleanup Kit (Bio-rad). The obtained protein was dissolved in 60 μL of swelling buffer (7 M urea, 2 M thiourea, 2% (w/v) CHAPS, 20 mM DTT, 0.2% (v/v) Biolyte 3-10, 0.039% (w/v) tris-(2-cyanoethyl)phosphine) to produce a protein solution for two-dimensional electrophoresis.

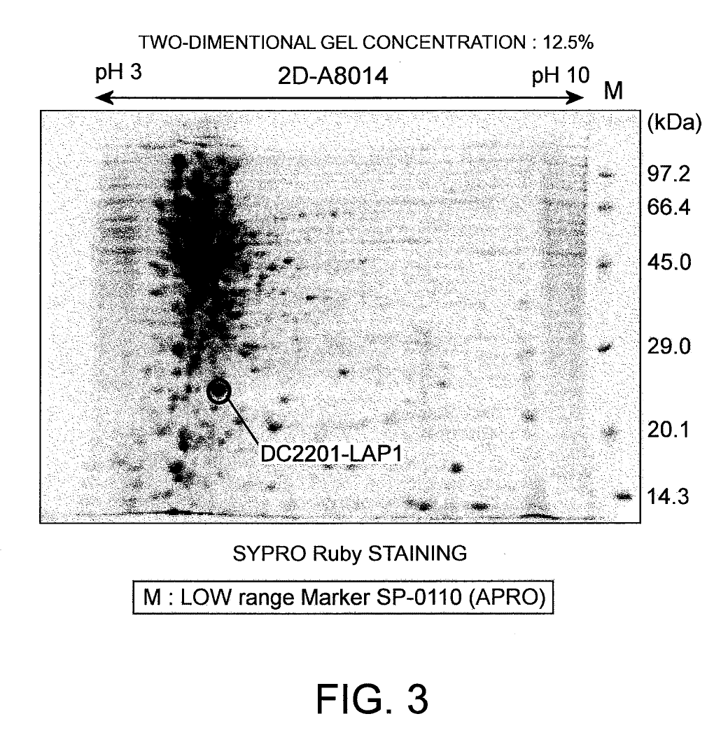

Two-dimensional electrophoresis was carried out by subjecting 50 μg of the protein to isoelectric focusing PROTEAN IEF Cell (Bio-rad). IPG ReadyStrip pH 3-10NL (7 cm, Bio-rad) was used for the isoelectric focusing electrophoresis gel. After the electrophoresis, the isoelectric focusing gel was removed, and this was subjected to SDS-PAGE using a 12.5% uniform gel. Thereafter, the gel was subjected to protein staining using SYPRO Ruby (Invitrogen), and then a large spot which appeared to be NBRC 103217-LAP1 was excised (FIG. 3).

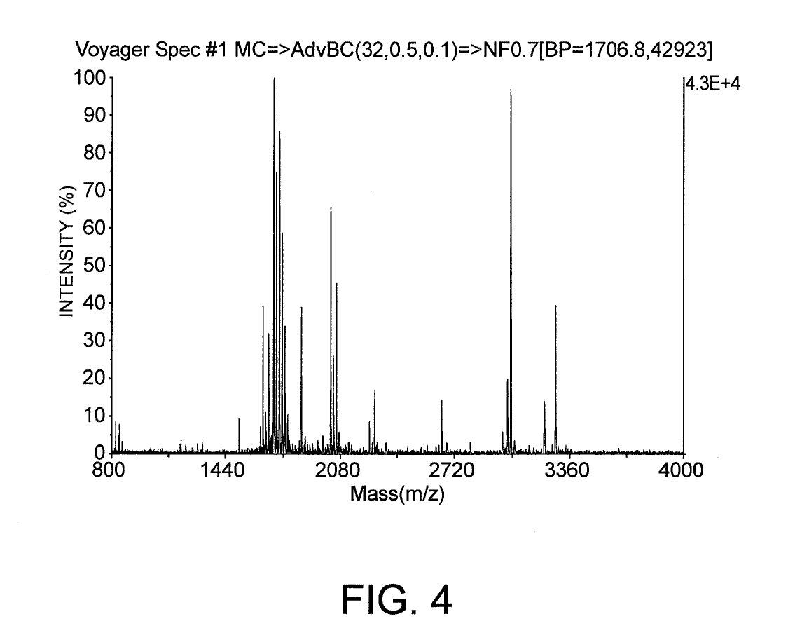

Tris-HCl (pH 8.0) containing trypsin was added to the excised gel slice, and this was incubated at 35° C. for 20 hours. After the obtained reaction solution was desalted using ZipTip C18 (Millipore), this was mixed with a matrix (α-cyano-4-hydroxyxinnamic acid solution), and spotted onto a plate. After air drying, this was subjected to MALDI-TOF MS. Using Voyager-DE STR (Applied Biosystems) in the reflector mode, peptides with cationic m/z values of 800 to 4,000 were detected (FIG. 4). Among proteins encoded by the ORFs of the NBRC 103217 strain, search for a protein predicted to be NBRC 103217-LAP1 was carried out using Mascot (Matrix science) based on the monoisotopic mass of the obtained peptides, and it is highly possible that the protein was KRH_{—}00400 encoding superoxide dismutase (SodA).

Therefore, the superoxide dismutase gene sodA (KRH 00400) was found to be highly expressed in the NBRC 103217 strain. Accordingly, when searching for a promoter predicted upstream of this gene, sequences “TTGACC” (SEQ ID NO: 15) and “GACAGT” (SEQ ID NO: 16) showing similarity to the −35 region and −10 region of an E. coli-derived promoter were found 67 bp to 62 bp and 43 bp to 38 bp upstream of the sodA gene start codon, respectively. To amplify DNA fragments containing these promoter regions by PCR, gene0045-F1 primer (5′-GGCGTCGACCGAGGGTAGGGCGGGAGTG-3′, SEQ ID NO: 17), gene0045-R1 primer (5′-TCGACTAGTGGCGTGATGGTTCAACTGTC-3′, SEQ ID NO: 18), and gene0045-R2-v2 primer (5′-AGCCATATGGAAACATCCTTTCGTTTGCG-3′, SEQ ID NO: 19) were prepared. Using 1 ng/μL of chromosomal DNA of the NBRC 103217 strain as the template, and either the gene0045-F1 and gene0045-R1 primer pair or the gene0045-F1 and gene0045-R2-v2 primer pair (0.2 μM each), PCR was performed by adding 25 U/mL of PrimeSTAR DNA Polymerase. The PCR was performed by incubation at 96° C. for five minutes, and then 30 cycles of a reaction consisting of three steps of: 98° C. for 10 seconds; 55° C. for 15 seconds; and 72° C. for thirty seconds, followed by incubation at 72° C. for ten minutes. The obtained DNA fragments (PsodA1 and PsodA2) were purified using the GFX PCR Purification Kit (GE Healthcare), and then they were double-digested with SalI-SpeI and NdeI-SalI, respectively. The obtained DNA fragments were ligated with fragments each produced by digesting pKV15358-8 (SEQ ID NO: 24), which is a vector for promoter evaluation, with the same restriction enzymes. E. coli JM109 was transformed using the reaction solution, and transformants carrying the plasmids of interest, pKV15358-5 (SEQ ID NO: 21) and pKV15358-6 (SEQ ID NO: 22), were obtained from the strains grown in an LB medium containing 50 μg/mL kanamycin. Using plasmid DNAs extracted from the obtained transformants, the NBRC 103217 strain was transformed, and by culturing at 30° C. on an SOB agar plate containing 400 μg/mL kanamycin, the NBRC 103217 strain transformants carrying each of the plasmids were obtained.

When each of the obtained transformants were cultured while shaking (150 rpm) in 100 mL of an LB medium without kanamycin and an LB medium containing 300 μg/mL kanamycin (500-mL baffled conical flask), the NBRC 103217 strain transformant that carries pKV15358-5 with optimized upstream and downstream sequences of the SD sequences showed a nitrilase activity 86 times greater than that of the NBRC 103217 strain transformant carrying pKV15358-4 which does not contain an sodA-derived promoter (P_{sodA}). Furthermore, in the NBRC 10317 strain, the transformant exhibited an activity three times greater than that of pKV15358-6, to which sodA's original upstream and downstream sequences of the SD sequence was added.

To produce high-expression vectors for the NBRC 103217 strain, shuttle vectors of the NBRC 103217 strain and E. coli, which are the BspHI-NdeI fragment (2,309 bp) of pKV15358-9 (SEQ ID NO: 25) and the BspHI-NdeI fragment (1,253 bp) of pKV15358-5, were ligated. The reaction solutions were used to transform E. coli JM109, and from the strains grown in an LB medium containing 50 μg/mL kanamycin, the plasmids of interest pKV15358-5 and pKV15358-7 (SEQ ID NO: 23) were obtained as high-expression vectors for the NBRC 103217 strain.

### INDUSTRIAL APPLICABILITY

By introducing shuttle vectors developed by the present inventors into microorganisms belonging to the genus Kocuria which can maintain their cellular structure in non-aqueous systems, expression of a foreign protein of interest can be induced in non-aqueous systems. Furthermore, by using microorganisms introduced with a shuttle vector of the present invention, the activity of a foreign protein (for example, catalyst activity) can be expressed in a non-aqueous system.

1. A vector capable of expressing a foreign protein in a microorganism, which comprises a polynucleotide comprising the nucleotide sequence of SEQ ID NO: 1, or a polynucleotide that hybridizes under stringent conditions with a complementary strand of a polynucleotide comprising the nucleotide sequence of SEQ ID NO: 1.

2. The vector of claim 1, wherein the vector additionally comprises a polynucleotide comprising the nucleotide sequence of SEQ ID NO: 2, or a polynucleotide that hybridizes under stringent conditions with a complementary strand of a polynucleotide comprising the nucleotide sequence of SEQ ID NO: 2.

3. The vector of claim 1 or 2, wherein the vector additionally comprises a polynucleotide comprising the nucleotide sequence of SEQ ID NO: 3 or 4, or a polynucleotide that hybridizes under stringent conditions with a complementary strand of a polynucleotide comprising the nucleotide sequence of SEQ ID NO: 3 or 4.

4. The vector of any one of claims 1 to 3, wherein the microorganism in which a foreign protein is expressed is a microorganism belonging to the genus Kocuria, the genus Micrococcus, or the genus Arthrobacter.

5. The vector of claim 4, wherein the microorganism belonging to the genus Kocuria is the Kocuria rhizophila NBRC 103217 strain.

6. A recombinant vector in which a DNA encoding a foreign protein is inserted into the vector of any one of claims 1 to 5.

7. A transformant in which a host is transformed with the vector of any one of claims 1 to 5 and the recombinant vector of claim 6.

8. A method for producing a foreign protein, which comprises the steps of culturing the transformant of claim 7 and isolating the foreign protein from the obtained culture.

9. A method for producing a substance of interest, wherein the transformant of claim 7 is used as a biocatalyst for producing the substance of interest in a non-aqueous reaction site.


SEQUENCE LISTING (25 sequences)

SEQ ID NO: 1 - DNA, 809 bp, Kocuria varians NBRC15358
tctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgc

SEQ ID NO: 2 - DNA, 20 bp, Kocuria varians NBRC15358
tctagcaaggaggccaacat

SEQ ID NO: 3 - DNA, 30 bp, Kocuria varians NBRC15358
ttgacctgcggaacggcggcgatggacagt

SEQ ID NO: 4 - DNA, 138 bp, Kocuria varians NBRC15358
gtcgaccgagggtagggcgggagtgggcgcggggaacgtggtgtgacggaacgtgaccggggcccgggagtcgtacgccagtggctgaacatccttgacctgcggaacggcggcgatggacagttgaaccatcacgcc

SEQ ID NO: 5 - DNA, 10913 bp, Kocuria varians NBRC15358
gaattccttgcattcaagaggacagaagccaaaacgcggacagggaatagcacgctacaggtgactcggatactgttctgccccaactgagcaatacattataatggttttgatgaaaaattacattcgccaaatccccgtggatggtcggacgccgccgatgagcgatgaggagttcgctgctgttgttgcccaggcaggcgatcccgccatgttcggggattggtacaccaaaagcgtgaaatcctccaatggtgcaggagaaggtgagtttactggccgcatctctattgacttgtctgattatctgaatgcgttctttcccggggaaattgaaaaacgcatcaaggcggttcaggagcagcttccgcatgttcccgattccgaaacggtaaggaaactagtcacagacgcactcaccccggacgggtggtcttctttggtgcggccgttctctatagcgcgcatccctgcctggagcacgtatttatatcgggtgcgccctggacttcacggccctgaagacatcaagactgtcggggacatctggacaccacccactggcacctcgccaggacgcgtcaaccgtcgaggtcaacccatcctttacacctcagcgcaatacccctccgtcgctttccgcgaatcgagagccaaggttggagaagtcgtcgcgcttagctgcttcgccataactgacgatctcaacctgctcaaccttgacgatatgagtccgcaggggaacttgaacaccagtcagcaacgcaaatggcggcagcttgcaaaattttaccggtgggccttccgggattcagggagcgacttgaatagtccacaacatcttgtaccccaagtattgactctggacttaaatatgctcatcccgcccctcgtcggttatggttatcaatccgtcattgtcaaccaccacgccgcgatcaacgtggccctagacgagcaaaaagcaatggatcacctacgcctggtaggcacgactgtgtggcgtattggcaagcccacaagcatgcttcttgcctccatggtaccgaaacatccgagacttactagagacacgccgctggtcccaggtaactttgcatggcctccagatcttgaagtcacccaaaggtcgtatcagagccgcgcatagccagcgtctggccatcgcacctcaccccatatataaatggggaggccagatgtagtaccgcggttcctgatggcccagccgtttttaccgagcaacccgctcgtctgatcctatgaggcgccgacgaactcagacggtcgctgtggagggcagatttcggatccgccggtagagagttgccctagatatcccgagatctcgagctacctgggcgactggatccccggactcgattaatcggacggcagtgcggatctgagaatcagtgaaagtttggcgacgtcccccgagatccttgccagcggccctacgtttctccaccgagtcagtgatccgctcacgcttaatctccaactccatctgtgccaacgcggccatcacggtgaacaccatcgaccccatcgggatcgatgtgtccacgtctcccccgccgaggttgagcacccgcaaccccacgccgcgctgccgaagttcttgagccagttccagcatggtctgagtggagcgccccaaccggtccaaggtcgtcaccaccagggtgtcgccctcgtggagagctcctacggcttggtcaaagcccgggcgggatgcccgggcccccgagaccccgtgatccacgtagaggtcatcccgacgcacccccgcgcccagcaggtctgtcacctgccggtccgtgccctggctcctcgtagacacccgcgcgtacccaatcaacttccccatgctctgtctccgcctcccgtatgtctcgcaaccgaccgtcagcaccccgatcggggcgaccggttgagagacaacgctacgagacagcccccgccccggatctccggcgccagtccaagacgccatgagacgtcttgcaacccattgattgcgagactcattccgtcgcgtctcgcgtctctcgcgcttggcacaggggacttggacaccgtgtaccactttgctttgcctgagctcatcgaggcggtcgcggcgacgggcaacgatgaggcgcagaccatgctcaccatgctgctcagcggtaagcgtctacgcgacattagcgacttgccgctcgacctggcggtctagcggcaagaatcagacgccgcaacacttcatggcctgagatcgccgcgggggctggtgcgccgatcgccgaccccaggtatcaaaacgcttgaagcaggggggtttcatgccaccccctgcaggggtgtcagaataggtgtcatgaccacctatggtgacacccccaacaacaaggtccctagccagggaggggtatcagggggttccagtgcggtgttcaccatgaaggaagccgccgatgtggcaggtgtcagcgtgagcaccctccgccgacggcgcgccgagctggaaggagcaggggccaccatcacctccgccgggtggcaggtgcccatgaccgcactcatcgccgtgggcctcatccacggcgaaggtttccataccgccccccaggccagaccatcgacgccgtctgcccagtccccggatgaagcaaagagcaccgggcgtctgctcgagcgcgtccgtgagttggaagctgaggtccaggaatggcgacggcgcgccgaagttgctgaagcacgagccgaagaacgccgccgagctctcgatgccctccagatcgccaacgaaaccgaacgcatagcgctgcggatgctcaccgggcagagctcccacacaccacaggctcctacggctcccgagcctgctcgtgccaggccggccgaggagccggaagcatccccgggacccaccacacccgtgacctctcaagagccgcgccgcgggttcttttcccgcatcttctcggcctaagagcttccgccgataggtgtggtggagcggtggcatgagacaggcacgagacctggtaaacacggatttgttgagaatcagtgtgttgacgaggagcactcgtgcctgccgttccatcgtgcatcatcgaacccatctgggaccagttccagatgctgatccctgctgtggcggacgagcatccattgggttgccaccggccacggatcccggaccgggtggtgttcgacaagctggtccaggtcctcgtccccggggccgcctacgagaggatctccgacacgacgtgttcggctaccacgatccgccgccggcgtgatgagtggatcgaggccggagcctttaaggagctggaacagttttgtctggagggctacgagaagatggtcgggctccagctggagaacttggccgtggacgggtgcatcaccaaggccccgtgcggtggcgaggccgccgggcggtcaccggtggaccgcggcaagcagggcacgaaacggtcgctgctggtcgagggcgccgggatcccgctgggtaccgtggtggccccggccaaccgccacgactccccgttgctgcgcccgacgttggagacgctgtccaggttcggtttcgaccggccggagcggatcaccgtgcacctggacgccgactacgactcgaagggcacccgggggttgctcgccgagctcggctgtgaagccaggatcgcggtcaagggcgtgcccgccccgatccagaacacgtccaggtgggtggtggaacggacgaactcctggcacaaccgcgggttccagaagctggccgtctgcaccgagaaacgtatccgggtaatcgaggcgttcatcgccctggccaacgccatcatcatcactcgcaggctcctccgcgaggcctggaacacccaccgctgggccacccgcccgacccgacgaccatggcctaatggcggaacctcttactcatcctccacggccaggcgcaggtggtcatcgccctacacggataccacaaggctttgccgcatgttggcaatggccaggcgccggtaaccagtcgcgagaagatcgtcgcttcccacggcggacagcgcacttttagacacctcctgcgttcgtcaaaagaagcggtccgggatcattcccggtttcagcccggaccgccgcgatccgtcgacgacgtcgagtgcttcaccctggcctggatcgacaggtacaacagcgcccgcccgcacacccgcctcggcaacgccccacccgccgagtacgaggcagcgcactgcgctgagcccaccaagtcatcgcgaccggcaatgacacctgcatagacgtggcatcaacgcgtagatgtttcagtttcttctgtgctggggctgtgtcaggtgctggaccgggtgtaggcagcggcgcgcaccacatcttcgtcctccagtccagctcctagtgctccgcccatggtggccaggctggcggtgagccagctgatcttcgcgtagtccaaagcgttcactgggtgaccggcgacaccggccagtacctgctcgtcgatgagcagcagcgcgccgacccaggacaggatgaataagacgaggtagaacaccacaaccccgatggccacggtcgcggtggtggccaaattgaacaggatcacctgctcacgttgggctcgccgccgtgggcgctcccacaagtcggctcccaggatcagggtggcactcaccgccccgatcgccatcaccgccagcaggaccaggcgcaccggcccgtaggccatggccagtacccataggtccgaggccaccaatgtcagcatgccggtggccgctgccacgaccagcgcccgggagagccgggccacgaagacccaaggctggttggcgcggatcatgcccagcagcaaccgcacgttgccgctcagcactcgggccgtgaactgcacgacattgtcgtccgggcgttcgtcgagatcggtggccagctggtgggctcttcgagcaagatcccgctgggcgttgaggtcttcggcgtcgtagcccaggagctgcccgaccacgtgcacggtggtctcttgcaccttctgccgcacgcgcacggctcccagggttgggacggagaccagtcctaccccgtgcacggggctgagctgggtgagcaccggacgtcggccggtcttcagggggatctcggtgagcaccacggccaaatcccagttctcctccagcacgcggtttcgggccgcctccagcaggtccaaggtctcggtgggtggatcgaccagccggtcttcggccatcttcagttgccagcgcactcccggataccgctgcaccaaaatcttgcgcaccgaggtggtgacctcggggccgagcaccgcactcagggatggtgagaccaccaggccgatgagcagctcggcatcggaaaggggttcggcaggatcggcggtggatcggcaacgctcagcagtcaccgctccatagtgaggcaaccacgggccggattgccactggctcgatggagcccccaaggtttcgtgcagcaccgcgggccggattcggtggtcgcctcgatcccggaaagagcggtgctgctcactgcgaaatcctctccccgccgacgccgccagcgcgaagacatcacagccagcgggcccgggacctgccggtgtcgaggacggcttctcgatgatgctgcagtgacggatcggttgaagtgcttcggcggtgtgggagcagtgatcagcgtggggccggtcgattccgttcggacaggtgatgcatcgactgcccagcggcaggtgcggtactgatgcgggggtacccggtgcaggggtgtcaaggcccagctccgaagacggcaagccgggcacgtgaaaggcgaattatggcccgccgcaacccgagcgggaaggcatgtgtggcacgaggcgccgaagacgggccgggcgtttactgcagagataaatatgcttatcctggtaaggtccgcgacatcttgtaaggccctgagagcgggcccgtcgctggtccaccacggaccgacgggctcggggacggggctgaggacccggagaggagccagtggcagaagtccacgaagatcgcaggcaccgcggctgaacggccgataccgagggggagcaagggaggccagggctgctgatgtgcccggactgttccgcgatttctcttcctaactttccgtccactcccgacccaggtgcccggcaaccccgggtgcaggccaccgtccgggtcggatcgtcaccacgaacaggaaacgataagcatgtcccatccctcccctcttccacccactgcttcgactcattaccaggtcctgcgcacagtgccctcctatgaccaggctcagcgccttgtcgatcgactctccgacgccgggttcccggttgagcacgtgcgcgtggtcggcaccgatctgcgcctggttgagcaggtcaccgggcggatgacctacggcaaggccgccctgtacggagccgcctccggggcctggctgggattgctcattggcttgctgttgggtcttttcacggtcgtggggtggctgtcggtaatcctgtgggcggtactcctgggcgcggtgtgggggctgatcttcgggttgctgggccacgcggccaccggcgggcgccgggacttcagtagcgtccagggcctggaggcgtcctcctacgagattctcgtggaggccgagtacctcgaggcggccagggccaagctcgacggccagccctgacagcaccgggcacgtggacgagaacgcccccaccggcgataccgggcccactccataaccgcaccccggcaccgaagagcccgccatcttcgacacgccccgacggggcgggggtagccccaccacctgcccccgccccgccctgggctgctggtgaatcggatccgccgggcccgcatcaccgcccgggtcgaagggtgcgagtaggccttgtcggccagcaccgcgtccgccgtgacgggccgggccggcccgtcacggcggacgctgatctgctccagcagcggcagcaactgtgggttgtccccggtctgtccctcggtcaggatgatgctgatcggcatcccggctccgtcgacggccaggtggatcttggaggtcagtcctccgcgggaccgtccgagacattctccggccacagcgatggcctcgaccggatcggagcagccccctttttccgggccccggcggcatgctggtgggcccggatggaggacgagtccacggagaacacccattcgacctccccggcagcgtcgtccttgaccaccgcctcatccaggatcttggccccagtgccgtccatggtccagatccgcagccgctcgtggccggtcttccacggcccataccgttcgggcacgtcccgccagggcgccccggtgcgcagcttccacaggatcccgttgaccacctgccggtgatcccgccaccgccggcccttaccgttggccggcagcaacggcgcgatgcgttcccaggccttgtccgtcagttctccacgcctcaccatgccccaggattaacaggacctgacggggattaacaaccccacgccgccgttccggggtacttagaggttgcgcgggtaggtcagggtcttcggcgggggcgtgtgtcccagcggtgagtggtccagacggtccggatcaggcgtcgaacgatgatgatggcgttggccagcgcgatccaggcgcgctggatcgcttcgctgcgatcggtgacgatctgcagggcacgaaagccgcgggtgtgccaggagttcgtgcgttcgaccttccagcggtgggtgtggttgaccggaacgaactgacccttcgggttgatctgccaggtgcagccgagctcatcgagtagctctcgagtgacggcggagtcgtagccggagtcgaggtggaccgtgatggtctcaggcaggtcgaacccgaacctgctcagccgctccaggatcgggcgcagcagcggtgagtcgtgccgattggcaccggcaagctcgcatccgatcgggacgccggcgccctcgacgagcagcgaacgtttcaggcccgacttcgcccggtcgaccggggacttgccagtgttgtccccgctgcacggggccttgacgatgcagccgtcgacgaccaggtcactcaggtccagtcccacgaccgtgtcgtaagcctccagcgcggcctgttcgagccggtcgaacacccccgcggcgatccactcgtcgcggcgggcccgcagcagggttgtggccgagacccggtcgtcggcgtgcctgtggtaagagccgcccagcacgagccgggccacgagcttgtcgaacaccacccggtccggtatccgccgccggtgacaccccaacggatgggtgtcgacccgcgcaggaatcagggcctcgaaacggctcgatgaacgatgctggcaccgcaggcacgggactcctcgcgacgacgtagatctaggcaatccacatcgaagcggacatcccgtgcctgcgtcacatccccgacaagccgcgccctacccgcgcgacctctaagcccagctacaggccccgcccacgatccccccggccggggccgggacgacgaaggtgctgcacctgctccaccgaatgaggtggggctacccactgctgctgttggggtgccggggactgtttccggggtgccgggttgtccagctccacgaacttgcggatctgctcccactccgcgcggcgggccgccaccacctgagcctgctgctcctgctgctgacgcaccgcgagcactgaacgtttgccgcgtcccgcctcacggcgccgcggtgcggtctgcaaaccgtgctccaactccagacgggtgcacgcgctttgggcggcgcggcggtcgttgcgaccgtgccacaccgtcccctcgtcactgacccggcacaccaccacatgcacgtggtcatcggcgtggcggaccatcacccacgggtgctccgcgaaccccatcgactccgcaaacgtctggcctgcctgcgcccactccgcatccgtcaaccgccgatccccagccgtgttgcgcaaagaggcctgccagatcggtttcgtgatctccgggcgggtgcgcatcgccgcgcgcatctccttcacccacgccttctcatccgcatccccggtcacgaacacgttcccgcccacgaccacaccaccggcataccggcccccgttgcgttcgtagaagtgctcattggcatgccccggcccatgcagataagccccgatgttgcccgggttcgccccccgggtgatcttcgcgatcacggcaactgcctccgcactactcccagctgactcgccagcgcgtccacggcctcatacagctgcggggtcgggacgacctgcccctggttcaccgcacgcatcaactggttcagattcgaccccacccgcgccagatccgcccggatcctcgccaactcctgcacctcacccatccgcggactctccgcatcaagctgttcctggactcgttcccgagcccacaccgacaaccgttgcccgccggcggcctccgcccacgcccggtgctcagcctcggtcaacgacatcgacacccgccgggtccgcggctccaccgccaccggccgcccaccagcaccccgcatccgaggcgccgacgctgtatcggttggttccgggtccggtgaggcgaggggctgctcgaggagcagcccctcccaccggttcccggaccccgacgggaccgtgcccggcgaggtcgaacgacgtgagaacaagcccaacgggaccaccctttcgagaccccgccaaggggtcgagaccagccctccgcaggagcaaggggattgaccggaacgggaacacctggcagcgaggaacgagcggtgcgccaggttttccgttccggacaatcataccttgcatccccgtttcagcgccccgacgaaggaggagaacagcgcgctggaacagggggcgtggtggtgtgctgggcctgaccgacgcaggaggacagagccagggccagggcaccaccacgcatcgtctgcagcgcagcggaagacgatcccccacctacccaaggtgggatgcgtgaacgaggttcttcctcggtcaccgaccagcccaaagggcctggtgttctagctgtggcggggaaaggagctcgcttcgctcgcgctcattcttttcctgccctccgctcttccccagtccgttctgtggtgctgttcaggagtggaccaaccggccagcctgggagggagggagggagcggagggcagggggatgccttaaacgttcccggggcacaccttcggtgaagaagggtggggtggggaacgtctaaggcatcttggaacacggtgcggatcagcccggtcggtcgatcggtcgcatcggttgaccacctcatgggtggttgaagaaggctcacggcattactcgtgggcagtggcggcatgtgcgcacagctccggtgctcgctgcgtggcagggctgctgtccacgagcggcactgtagggcttggcactccttcgctatccaggtcttgcgatccctggtgacggcccccgtctgtgcgctcggcaaaaggaaccggctctgacccggggagcaccgcgtgtaaggcggctggtgatgttgatacggggccgcgtctcaggccccacccaggtcttccatcgctgggcgctccattggcacccgttggcgcaggcgctagaatggactcactgtttcagttcaacttcaggatagggcaccgttggcgcggtgcccttccgcatgtccagggcgtgtcgccagacacgcggtctgcgggccaccccgttgtaggtggcccgcagaccgctctagatactcttcccgtcactgcgtcggagtggccgctagcccttcactgatctggga

SEQ ID NO: 6 - DNA, 4303 bp, Artificial
  An artificially synthesized vector sequence
tctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgctgcagacgatgcgtggtggtgccctggccctggctctgtcctcctgcgtcggtcaggcccagcacaccaccacgccccctgttccagcgcgctgttctcctccttcgtcggggcgctgaaacggggatgcaaggtatgattgtccggaacggaaaacctggcgcaccgctcgttcctcgctgccaggtgttcccgttccggtcaatccccttgctcctgcggagggctggtctcgaccccttggcggggtctcgaaagggtggtcccgttgggcttgttctcacgtcgttcgacctcgccgggcacggtcccgtcggggtccgggaaccggtgggaggggctgctcctcgagcagcccctcgcctcaccggacccggaaccaaccgatacagcgtcggcgcctcggatgcggggtgctggtgggcggccggtggcggtggagccgcggacccggcgggtgtcgatgtcgttgaccgaggctgagcaccgggcgtgggcggaggccgccggcgggcaacggttgtcggtgtgggctcgggaacgagtccaggaacagcttgatgcggagagtccgcggatgggtgaggtgcaggagttggcgaggatccgggcggatctggcgcgggtggggtcgaatctgaaccagttgatgcgtgcggtgaaccaggggcaggtcgtcccgaccccgcagctgtatgaggccgtggacgcgctggcgagtcagctgggagtagtgcggaggcagttgccgtgatcgcgaagatcacccggggggcgaacccgggcaacatcggggcttatctgcatgggccggggcatgccaatgagcacttctacgaacgcaacgggggccggtatgccggtggtgtggtcgtgggcgggaacgtgttcgtgaccggggatgcggatgagaaggcgtgggtgaaggagatgcgcgcggcgatgcgcacccgcccggagatcacgaaaccgatctggcaggcctctttgcgcaacacggctggggatcggcggttgacggatgcggagtgggcgcaggcaggccagacgtttgcggagtcgatggggttcgcggagcacccgtgggtgatggtccgccacgccgatgaccacgtgcatgtggtggtgtgccgggtcagtgacgaggggacggtgtggcacggtcgcaacgaccgccgcgccgcccaaagcgcgtgcacccgtctggagttggagcacggtttgcagaccgcaccgcggcgccgtgaggcgggacgcggcaaacgttcagtgctcgcggtgcgtcagcagcaggagcagcaggctcaggtggtggcggcccgccgcgcggagtgggagcagatccgcaagttcgtggagctggacaacccggcaccccggaaacagtccccggcaccccaacagcagcagtgggtagccccacctcattcggtggagcaggtgcagcaccttcgtcgtcccggccccggccggggggatcgtgggcggggcctgtagctgggcttagaggtcgcgcgggtagggcgcggcttgtcggggatgtgacgcagctgtctcttatacacatctcaaccatcatcgatgaattgcttcgttaatacagatgtaggtgttccacagggtagccagcagcatcctgcgatgcagatccggatgccatttcattacctctttctccgcacccgacatagatccgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatctgccatttcattacctctttctccgcacccgacatagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagggtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatcc

SEQ ID NO: 7 - DNA, 4326 bp, Artificial
  An artificially synthesized vector sequence
tctagcaaggaggccaacatatgtctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgctgcagacgatgcgtggtggtgccctggccctggctctgtcctcctgcgtcggtcaggcccagcacaccaccacgccccctgttccagcgcgctgttctcctccttcgtcggggcgctgaaacggggatgcaaggtatgattgtccggaacggaaaacctggcgcaccgctcgttcctcgctgccaggtgttcccgttccggtcaatccccttgctcctgcggagggctggtctcgaccccttggcggggtctcgaaagggtggtcccgttgggcttgttctcacgtcgttcgacctcgccgggcacggtcccgtcggggtccgggaaccggtgggaggggctgctcctcgagcagcccctcgcctcaccggacccggaaccaaccgatacagcgtcggcgcctcggatgcggggtgctggtgggcggccggtggcggtggagccgcggacccggcgggtgtcgatgtcgttgaccgaggctgagcaccgggcgtgggcggaggccgccggcgggcaacggttgtcggtgtgggctcgggaacgagtccaggaacagcttgatgcggagagtccgcggatgggtgaggtgcaggagttggcgaggatccgggcggatctggcgcgggtggggtcgaatctgaaccagttgatgcgtgcggtgaaccaggggcaggtcgtcccgaccccgcagctgtatgaggccgtggacgcgctggcgagtcagctgggagtagtgcggaggcagttgccgtgatcgcgaagatcacccggggggcgaacccgggcaacatcggggcttatctgcatgggccggggcatgccaatgagcacttctacgaacgcaacgggggccggtatgccggtggtgtggtcgtgggcgggaacgtgttcgtgaccggggatgcggatgagaaggcgtgggtgaaggagatgcgcgcggcgatgcgcacccgcccggagatcacgaaaccgatctggcaggcctctttgcgcaacacggctggggatcggcggttgacggatgcggagtgggcgcaggcaggccagacgtttgcggagtcgatggggttcgcggagcacccgtgggtgatggtccgccacgccgatgaccacgtgcatgtggtggtgtgccgggtcagtgacgaggggacggtgtggcacggtcgcaacgaccgccgcgccgcccaaagcgcgtgcacccgtctggagttggagcacggtttgcagaccgcaccgcggcgccgtgaggcgggacgcggcaaacgttcagtgctcgcggtgcgtcagcagcaggagcagcaggctcaggtggtggcggcccgccgcgcggagtgggagcagatccgcaagttcgtggagctggacaacccggcaccccggaaacagtccccggcaccccaacagcagcagtgggtagccccacctcattcggtggagcaggtgcagcaccttcgtcgtcccggccccggccggggggatcgtgggcggggcctgtagctgggcttagaggtcgcgcgggtagggcgcggcttgtcggggatgtgacgcagctgtctcttatacacatctcaaccatcatcgatgaattgcttcgttaatacagatgtaggtgttccacagggtagccagcagcatcctgcgatgcagatccggatgccatttcattacctctttctccgcacccgacatagatccgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatctgccatttcattacctctttctccgcacccgacatagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagggtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatcc

SEQ ID NO: 8 - DNA, 5 bp, Artificial
  An artificially synthesized nucleotide sequence
aacct

SEQ ID NO: 9 - RNA, 14 nt, Artificial
  An artificially synthesized nucleotide sequence
aucaccuccuuucu

SEQ ID NO: 10 - RNA, 6 nt, Artificial
  An artificially synthesized nucleotide sequence
aggagg

SEQ ID NO: 11 - DNA, 19 bp, Artificial
  An artificially synthesized nucleotide sequence
ctagcaaggaggccaacct

SEQ ID NO: 12 - DNA, 22 bp, Artificial
  An artificially synthesized nucleotide sequence
ctagcaaggaggccaacatatg

SEQ ID NO: 13 - DNA, 36 bp, Artificial
  An artificially synthesized nucleotide sequence
gtgcatatgagcgagtacacccagcaataccgcgtc

SEQ ID NO: 14 - DNA, 37 bp, Artificial
  An artificially synthesized nucleotide sequence
tcctctagatcagagctgaacggcctcctccgaacgc

SEQ ID NO: 15 - DNA, 6 bp, Artificial
  An artificially synthesized nucleotide sequence
ttgacc

SEQ ID NO: 16 - DNA, 6 bp, Artificial
  An artificially synthesized nucleotide sequence
gacagt

SEQ ID NO: 17 - DNA, 28 bp, Artificial
  An artificially synthesized nucleotide sequence
ggcgtcgaccgagggtagggcgggagtg

SEQ ID NO: 18 - DNA, 29 bp, Artificial
  An artificially synthesized nucleotide sequence
tcgactagtggcgtgatggttcaactgtc

SEQ ID NO: 19 - DNA, 29 bp, Artificial
  An artificially synthesized nucleotide sequence
agccatatggaaacatcctttcgtttgcg

SEQ ID NO: 20 - DNA, 5445 bp, Artificial
  An artificially synthesized vector sequence
tctagcaaggaggccaacatatgagcgagtacacccagcaataccgcgtcgccgtcgtgcaggccgagccggtctggaacgacgtggcaggcggcatcgagaagaccatccggctggccaggcaggcagcagagggcggcgccgacctcatcgcgttccccgaggtgtggatccccggctacccgtggttcctctggctggactccgtggcgtggcagtcccggttcgtgctgccgtacatggagaactcgatcgagctcggcagcgcggagcaccgcgagctggagcgcgcagcggcggaggtcgggatcgcgatgagcctcggcttcagcgagcgggatcgaggttccctgtacatcgcgcaggcgttcatcgacagcgacggcgtgaccaggaccacccggcgcaagctgaagccgacgcacgtggagcgcacgctgttcggggagggcgacggcgccgatatccaggtgatcgacaccgacttcggccggctcggccagctcaactgctgggagcacctgcagccgctggcgaagtacgccatgttctcgcagggtgagcaggtccacgtggccgcgtggccgagcttctccatcttcgaggatgccgtctacgccctcggtccagaggtgaacgtgggggcggcacggcagtacgcagtggagggccagtgcttcgtgctctcaccgtgcgggatggtggggcaggcgggacaggaactcctggccgacaccgagctgaagaaggacctgctgcggctcggcggtgggcatgcgcgcatcttcggccccgacggccgccccctggccgagcccctcgcccccgatcaggaggggctgctcttcgccgacctggaccatcgcgccatcctggccgccaagaacgcagcggaccccgtggggcactactcgcgccccgacgtgttccggctgcacttcgacaacacggccaagcccatggtggtggagggtgccatggcggccggcggtgccgacgagagggatgaagccgacggcatcgcagccgagggcatcgtgcccacccgcagctctgcgcccggtgatggcgatggcgtcggggcggacgacggcgctgcgtcgcacgccggcaggcgttcggaggaggccgttcagctctgatctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgctgcagacgatgcgtggtggtgccctggccctggctctgtcctcctgcgtcggtcaggcccagcacaccaccacgccccctgttccagcgcgctgttctcctccttcgtcggggcgctgaaacggggatgcaaggtatgattgtccggaacggaaaacctggcgcaccgctcgttcctcgctgccaggtgttcccgttccggtcaatccccttgctcctgcggagggctggtctcgaccccttggcggggtctcgaaagggtggtcccgttgggcttgttctcacgtcgttcgacctcgccgggcacggtcccgtcggggtccgggaaccggtgggaggggctgctcctcgagcagcccctcgcctcaccggacccggaaccaaccgatacagcgtcggcgcctcggatgcggggtgctggtgggcggccggtggcggtggagccgcggacccggcgggtgtcgatgtcgttgaccgaggctgagcaccgggcgtgggcggaggccgccggcgggcaacggttgtcggtgtgggctcgggaacgagtccaggaacagcttgatgcggagagtccgcggatgggtgaggtgcaggagttggcgaggatccgggcggatctggcgcgggtggggtcgaatctgaaccagttgatgcgtgcggtgaaccaggggcaggtcgtcccgaccccgcagctgtatgaggccgtggacgcgctggcgagtcagctgggagtagtgcggaggcagttgccgtgatcgcgaagatcacccggggggcgaacccgggcaacatcggggcttatctgcatgggccggggcatgccaatgagcacttctacgaacgcaacgggggccggtatgccggtggtgtggtcgtgggcgggaacgtgttcgtgaccggggatgcggatgagaaggcgtgggtgaaggagatgcgcgcggcgatgcgcacccgcccggagatcacgaaaccgatctggcaggcctctttgcgcaacacggctggggatcggcggttgacggatgcggagtgggcgcaggcaggccagacgtttgcggagtcgatggggttcgcggagcacccgtgggtgatggtccgccacgccgatgaccacgtgcatgtggtggtgtgccgggtcagtgacgaggggacggtgtggcacggtcgcaacgaccgccgcgccgcccaaagcgcgtgcacccgtctggagttggagcacggtttgcagaccgcaccgcggcgccgtgaggcgggacgcggcaaacgttcagtgctcgcggtgcgtcagcagcaggagcagcaggctcaggtggtggcggcccgccgcgcggagtgggagcagatccgcaagttcgtggagctggacaacccggcaccccggaaacagtccccggcaccccaacagcagcagtgggtagccccacctcattcggtggagcaggtgcagcaccttcgtcgtcccggccccggccggggggatcgtgggcggggcctgtagctgggcttagaggtcgcgcgggtagggcgcggcttgtcggggatgtgacgcagctgtctcttatacacatctcaaccatcatcgatgaattgcttcgttaatacagatgtaggtgttccacagggtagccagcagcatcctgcgatgcagatccggatgccatttcattacctctttctccgcacccgacatagatccgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatctgccatttcattacctctttctccgcacccgacatagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagggtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatcc

SEQ ID NO: 21 - DNA, 4681 bp, Artificial
  An artificially synthesized vector sequence
tcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatctcgaggtcgaccgagggtagggcgggagtgggcgcggggaacgtggtgtgacggaacgtgaccggggcccgggagtcgtacgccagtggctgaacatccttgacctgcggaacggcggcgatggacagttgaaccatcacgccactagttctaggatcctctagcaaggaggccaacatatgagcgagtacacccagcaataccgcgtcgccgtcgtgcaggccgagccggtctggaacgacgtggcaggcggcatcgagaagaccatccggctggccaggcaggcagcagagggcggcgccgacctcatcgcgttccccgaggtgtggatccccggctacccgtggttcctctggctggactccgtggcgtggcagtcccggttcgtgctgccgtacatggagaactcgatcgagctcggcagcgcggagcaccgcgagctggagcgcgcagcggcggaggtcgggatcgcgatgagcctcggcttcagcgagcgggatcgaggttccctgtacatcgcgcaggcgttcatcgacagcgacggcgtgaccaggaccacccggcgcaagctgaagccgacgcacgtggagcgcacgctgttcggggagggcgacggcgccgatatccaggtgatcgacaccgacttcggccggctcggccagctcaactgctgggagcacctgcagccgctggcgaagtacgccatgttctcgcagggtgagcaggtccacgtggccgcgtggccgagcttctccatcttcgaggatgccgtctacgccctcggtccagaggtgaacgtgggggcggcacggcagtacgcagtggagggccagtgcttcgtgctctcaccgtgcgggatggtggggcaggcgggacaggaactcctggccgacaccgagctgaagaaggacctgctgcggctcggcggtgggcatgcgcgcatcttcggccccgacggccgccccctggccgagcccctcgcccccgatcaggaggggctgctcttcgccgacctggaccatcgcgccatcctggccgccaagaacgcagcggaccccgtggggcactactcgcgccccgacgtgttccggctgcacttcgacaacacggccaagcccatggtggtggagggtgccatggcggccggcggtgccgacgagagggatgaagccgacggcatcgcagccgagggcatcgtgcccacccgcagctctgcgcccggtgatggcgatggcgtcggggcggacgacggcgctgcgtcgcacgccggcaggcgttcggaggaggccgttcagctctgatctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgccctctttgcgcaacacggctggggatcggcggttgacggatgcggagtgggcgcaggcaggccagacgtttgcggagtcgatggggttcgcggagcacccgtgggtgatggtccgccacgccgatgaccacgtgcatgtggtggtgtgccgggtcagtgacgaggggacggtgtggcacggtcgcaacgaccgccgcgccgcccaaagcgcgtgcacccgtctggagttggagcacggtttgcagaccgcaccgcggcgccgtgaggcgggacgcggcaaacgttcagtgctcgcggtgcgtcagcagcaggagcagcaggctcaggtggtggcggcccgccgcgcggagtgggagcagatccgcaagttcgtggagctggacaacccggcaccccggaaacagtccccggcaccccaacagcagcagtgggtagccccacctcattcggtggagcaggtgcagcaccttcgtcgtcccggccccggccggggggatcgtgggcggggcctgtagctgggcttagaggtcgcgcgggtagggcgcggcttgtcggggatgtgacgcagctgtctcttatacacatctcaaccatcatcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttgg

SEQ ID NO: 22 - DNA, 4668 bp, Artificial
  An artificially synthesized vector sequence
tcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatctcgaggtcgaccgagggtagggcgggagtgggcgcggggaacgtggtgtgacggaacgtgaccggggcccgggagtcgtacgccagtggctgaacatccttgacctgcggaacggcggcgatggacagttgaaccatcacgcccgcaaacgaaaggatgtttccatatgagcgagtacacccagcaataccgcgtcgccgtcgtgcaggccgagccggtctggaacgacgtggcaggcggcatcgagaagaccatccggctggccaggcaggcagcagagggcggcgccgacctcatcgcgttccccgaggtgtggatccccggctacccgtggttcctctggctggactccgtggcgtggcagtcccggttcgtgctgccgtacatggagaactcgatcgagctcggcagcgcggagcaccgcgagctggagcgcgcagcggcggaggtcgggatcgcgatgagcctcggcttcagcgagcgggatcgaggttccctgtacatcgcgcaggcgttcatcgacagcgacggcgtgaccaggaccacccggcgcaagctgaagccgacgcacgtggagcgcacgctgttcggggagggcgacggcgccgatatccaggtgatcgacaccgacttcggccggctcggccagctcaactgctgggagcacctgcagccgctggcgaagtacgccatgttctcgcagggtgagcaggtccacgtggccgcgtggccgagcttctccatcttcgaggatgccgtctacgccctcggtccagaggtgaacgtgggggcggcacggcagtacgcagtggagggccagtgcttcgtgctctcaccgtgcgggatggtggggcaggcgggacaggaactcctggccgacaccgagctgaagaaggacctgctgcggctcggcggtgggcatgcgcgcatcttcggccccgacggccgccccctggccgagcccctcgcccccgatcaggaggggctgctcttcgccgacctggaccatcgcgccatcctggccgccaagaacgcagcggaccccgtggggcactactcgcgccccgacgtgttccggctgcacttcgacaacacggccaagcccatggtggtggagggtgccatggcggccggcggtgccgacgagagggatgaagccgacggcatcgcagccgagggcatcgtgcccacccgcagctctgcgcccggtgatggcgatggcgtcggggcggacgacggcgctgcgtcgcacgccggcaggcgttcggaggaggccgttcagctctgatctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgccctctttgcgcaacacggctggggatcggcggttgacggatgcggagtgggcgcaggcaggccagacgtttgcggagtcgatggggttcgcggagcacccgtgggtgatggtccgccacgccgatgaccacgtgcatgtggtggtgtgccgggtcagtgacgaggggacggtgtggcacggtcgcaacgaccgccgcgccgcccaaagcgcgtgcacccgtctggagttggagcacggtttgcagaccgcaccgcggcgccgtgaggcgggacgcggcaaacgttcagtgctcgcggtgcgtcagcagcaggagcagcaggctcaggtggtggcggcccgccgcgcggagtgggagcagatccgcaagttcgtggagctggacaacccggcaccccggaaacagtccccggcaccccaacagcagcagtgggtagccccacctcattcggtggagcaggtgcagcaccttcgtcgtcccggccccggccggggggatcgtgggcggggcctgtagctgggcttagaggtcgcgcgggtagggcgcggcttgtcggggatgtgacgcagctgtctcttatacacatctcaaccatcatcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttgg

SEQ ID NO: 23 - DNA, 3562 bp, Artificial
  An artificially synthesized vector sequence
tcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatctcgaggtcgaccgagggtagggcgggagtgggcgcggggaacgtggtgtgacggaacgtgaccggggcccgggagtcgtacgccagtggctgaacatccttgacctgcggaacggcggcgatggacagttgaaccatcacgccactagttctaggatcctctagcaaggaggccaacatatgtctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgccctctttgcgcaacacggctggggatcggcggttgacggatgcggagtgggcgcaggcaggccagacgtttgcggagtcgatggggttcgcggagcacccgtgggtgatggtccgccacgccgatgaccacgtgcatgtggtggtgtgccgggtcagtgacgaggggacggtgtggcacggtcgcaacgaccgccgcgccgcccaaagcgcgtgcacccgtctggagttggagcacggtttgcagaccgcaccgcggcgccgtgaggcgggacgcggcaaacgttcagtgctcgcggtgcgtcagcagcaggagcagcaggctcaggtggtggcggcccgccgcgcggagtgggagcagatccgcaagttcgtggagctggacaacccggcaccccggaaacagtccccggcaccccaacagcagcagtgggtagccccacctcattcggtggagcaggtgcagcaccttcgtcgtcccggccccggccggggggatcgtgggcggggcctgtagctgggcttagaggtcgcgcgggtagggcgcggcttgtcggggatgtgacgcagctgtctcttatacacatctcaaccatcatcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttgg

SEQ ID NO: 24 - DNA, 4594 bp, Artificial
  An artificially synthesized vector sequence
tcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatctcgaggtcgacggtatcgataagcttgatatcgaattcctgcagcccgggggatccactagttctaggatcctctagcaaggaggccaacatatgagcgagtacacccagcaataccgcgtcgccgtcgtgcaggccgagccggtctggaacgacgtggcaggcggcatcgagaagaccatccggctggccaggcaggcagcagagggcggcgccgacctcatcgcgttccccgaggtgtggatccccggctacccgtggttcctctggctggactccgtggcgtggcagtcccggttcgtgctgccgtacatggagaactcgatcgagctcggcagcgcggagcaccgcgagctggagcgcgcagcggcggaggtcgggatcgcgatgagcctcggcttcagcgagcgggatcgaggttccctgtacatcgcgcaggcgttcatcgacagcgacggcgtgaccaggaccacccggcgcaagctgaagccgacgcacgtggagcgcacgctgttcggggagggcgacggcgccgatatccaggtgatcgacaccgacttcggccggctcggccagctcaactgctgggagcacctgcagccgctggcgaagtacgccatgttctcgcagggtgagcaggtccacgtggccgcgtggccgagcttctccatcttcgaggatgccgtctacgccctcggtccagaggtgaacgtgggggcggcacggcagtacgcagtggagggccagtgcttcgtgctctcaccgtgcgggatggtggggcaggcgggacaggaactcctggccgacaccgagctgaagaaggacctgctgcggctcggcggtgggcatgcgcgcatcttcggccccgacggccgccccctggccgagcccctcgcccccgatcaggaggggctgctcttcgccgacctggaccatcgcgccatcctggccgccaagaacgcagcggaccccgtggggcactactcgcgccccgacgtgttccggctgcacttcgacaacacggccaagcccatggtggtggagggtgccatggcggccggcggtgccgacgagagggatgaagccgacggcatcgcagccgagggcatcgtgcccacccgcagctctgcgcccggtgatggcgatggcgtcggggcggacgacggcgctgcgtcgcacgccggcaggcgttcggaggaggccgttcagctctgatctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgccctctttgcgcaacacggctggggatcggcggttgacggatgcggagtgggcgcaggcaggccagacgtttgcggagtcgatggggttcgcggagcacccgtgggtgatggtccgccacgccgatgaccacgtgcatgtggtggtgtgccgggtcagtgacgaggggacggtgtggcacggtcgcaacgaccgccgcgccgcccaaagcgcgtgcacccgtctggagttggagcacggtttgcagaccgcaccgcggcgccgtgaggcgggacgcggcaaacgttcagtgctcgcggtgcgtcagcagcaggagcagcaggctcaggtggtggcggcccgccgcgcggagtgggagcagatccgcaagttcgtggagctggacaacccggcaccccggaaacagtccccggcaccccaacagcagcagtgggtagccccacctcattcggtggagcaggtgcagcaccttcgtcgtcccggccccggccggggggatcgtgggcggggcctgtagctgggcttagaggtcgcgcgggtagggcgcggcttgtcggggatgtgacgcagctgtctcttatacacatctcaaccatcatcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttgg

SEQ ID NO: 25 - DNA, 3404 bp, Artificial
  An artificially synthesized vector sequence
tcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatcctctagcaaggaggccaacatatgtctagagcggtctgcgggccacctacaacggggtggcccgcagaccgcgtgtctggcgacacgccctggacatgcggaagggcaccgcgccaacggtgccctatcctgaagttgaactgaaacagtgagtccattctagcgcctgcgccaacgggtgccaatggagcgcccagcgatggaagacctgggtggggcctgagacgcggccccgtatcaacatcaccagccgccttacacgcggtgctccccgggtcagagccggttccttttgccgagcgcacagacgggggccgtcaccagggatcgcaagacctggatagcgaaggagtgccaagccctacagtgccgctcgtggacagcagccctgccacgcagcgagcaccggagctgtgcgcacatgccgccactgcccacgagtaatgccgtgagccttcttcaaccacccatgaggtggtcaaccgatgcgaccgatcgaccgaccgggctgatccgcaccgtgttccaagatgccttagacgttccccaccccacccttcttcaccgaaggtgtgccccgggaacgtttaaggcatccccctgccctccgctccctccctccctcccaggctggccggttggtccactcctgaacagcaccacagaacggactggggaagagcggagggcaggaaaagaatgagcgcgagcgaagcgagctcctttccccgccacagctagaacaccaggccctttgggctggtcggtgaccgaggaagaacctcgttcacgcatcccaccttgggtaggtgggggatcgtcttccgctgcgccctctttgcgcaacacggctggggatcggcggttgacggatgcggagtgggcgcaggcaggccagacgtttgcggagtcgatggggttcgcggagcacccgtgggtgatggtccgccacgccgatgaccacgtgcatgtggtggtgtgccgggtcagtgacgaggggacggtgtggcacggtcgcaacgaccgccgcgccgcccaaagcgcgtgcacccgtctggagttggagcacggtttgcagaccgcaccgcggcgccgtgaggcgggacgcggcaaacgttcagtgctcgcggtgcgtcagcagcaggagcagcaggctcaggtggtggcggcccgccgcgcggagtgggagcagatccgcaagttcgtggagctggacaacccggcaccccggaaacagtccccggcaccccaacagcagcagtgggtagccccacctcattcggtggagcaggtgcagcaccttcgtcgtcccggccccggccggggggatcgtgggcggggcctgtagctgggcttagaggtcgcgcgggtagggcgcggcttgtcggggatgtgacgcagctgtctcttatacacatctcaaccatcatcgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttgg